(19) United States
(12) Patent Application Publication
(10) Pub. No.: US 2014/0366906 A1
(43) Pub. Date: Dec. 18, 2014

(54) HAIR COLOUR COMPOSITION

(71) Applicant: Conopco, Inc., d/b/a UNILEVER, Englewood Cliffs, NJ (US)

(72) Inventors: Ranjit Kaur Bhogal, Huntingdon (GB); John Casey, Bedford (GB); Karl John Hunter, Bedford (GB)

(73) Assignee: Conopco, Inc., d/b/a UNILEVER, Englewood Cliffs, NJ (US)

(21) Appl. No.: 14/362,248

(22) PCT Filed: Dec. 3, 2012
(86) PCT No.: PCT/EP2012/074204

§ 371 Date: Jun. 2, 2014

(30) Foreign Application Priority Data
Dec. 6, 2011 (EP) 11192189

(51) Int. Cl. A61K 8/49; A61K 8/22; A61Q 5/10

(52) U.S. Cl. CPC A61K 8/49; A61Q 5/10; A61K 8/22
USPC 132/208; 8/406; 132/314

(57) ABSTRACT

The invention relates to a hair colour composition, in particular a hair colour composition comprising a flavonoid, a catalyst consisting of iron (II) or cobalt (II) complexed to a non-proteinaceous heterocyclic macrocycle ligand composed of pyrrole or pyrroline subunits, and either a hydrogen peroxide generator or 0.0001 to less than 1.5% w/w hydrogen peroxide. The flavonoid is selected from the group consisting of flavones, isoflavones, flavans, isoflavans, flavanones, flavonols, flavan-3-ols, dihydroflavon ol, flavanonols, proanthocyanidins, aurones, chalcones, dihydrochalcones, flavonolignans, and derivatives thereof, wherein the hair colour composition has a pH of 4.5 to 8.0, preferably 5.5 to 7.5.

The invention relates to a hair colour composition, in particular a hair colour composition comprising a flavonoid, a catalyst consisting of a metal ion complexed to a non-proteinaceous heterocyclic macrocycle ligand composed of pyrrole or pyrroline subunits, and either a hydrogen peroxide generator or 0.0001 to less than 1.5% w/w hydrogen peroxide. The flavonoid is selected from the group consisting of flavones, isoflavones, flavans, isoflavans, flavanones, flavonols, flavan-3-ols, dihydroflavonol, flavanonols, proanthocyanidins, aurones, chalcones, dihydrochalcones, flavonolignans, and derivatives thereof.

WO 03/047542 (Unilever) discloses a hair colouring composition comprising a first composition which comprises a transition metal salt or complex which is first applied to the hair and a second composition which comprises the following two compositions which are mixed just prior to application to the hair: (a) a composition comprising a water-soluble peroxygen oxidizing agent and (b) a composition optionally comprising one or more oxidative hair colouring agents selected from the group consisting of an aromatic diamine, an aminophenol, a polyhydric phenol, a catechol and mixtures thereof. Examples of a transition metal salt or complex include metallo-porphyrin catalysts but preferred is one formed from a manganese salt and 1,4,7-trimethyl-1,4,7-triazocyclononane. An example of a water-soluble peroxygen oxidizing agent is hydrogen peroxide.

US 2007/0226918 (Tagawa) discloses a composition and kit for hair dyeing comprising a first liquid containing an alkaline ingredient and an oxidation dye and a second liquid containing an oxidant wherein the improvement being the addition of a chlorophyll derivative, such as sodium iron chlorophyllin, into the first liquid. Hydrogen peroxide is mentioned as a preferred oxidant and it is preferable to adjust the pH of the mixture of the first liquid and the second liquid to be effectively neutral-alkalescence in the range of 6.0-8.6. p-phenyl diamine, toluenediamine and p-aminophenol are mentioned as dyestuff medium and m-phenylene, resorcin, diaminopyridine, naphthol and amino cresol are mentioned as coupling agents. An anti-inflammatory agent, such as “Phytoblend Tips” (Ichimaru Pharcos Co. Ltd.) containing extracts of comfrey, linden and peony, can be used. All of the aforementioned dyestuff mediums and coupling agents are allergenic.

Huang et al (Chinese J. of Anal. Chem., 29, 4, 378-382 (2001)) discloses that natural enzymes are expensive, and prone to deactivation and denaturation and are thus difficult to preserve. Studies on the development and application of “artificial enzymes” or “mimic enzymes” have achieved some success through mimicking catalytic properties of horse radish peroxidase by using metalloporphyrins, metal phthalocyanines, Schiff-base complexes, or some small bio-molecules. These mimic enzymes, however, can only mimic catalytic sites of natural enzymes, and lack the binding sites and regulating sites of the natural enzymes, which means that their catalytic activity and specificity are unsatisfactory.

Huang et al (Chinese J. of Anal. Chem., 29, 4, 378-382 (2001)) then discloses that haemoglobin and horse radish peroxidase contain the same ferriporphyrin prosthetic group and that studies on a novel method for determination of hydrogen peroxide and indirect determination of glucose through spectrophotometry using the enzyme catalytic properties of the hydrogen peroxide-4-aminoantipyrine-2,3,4-trichlorophenol reaction system with haemoglobin as an enzyme mimetic of peroxidase found that haemoglobin possesses the typical properties of peroxidase and high enzyme catalytic activity.

Further studies by Huang et al (Anal. Letters, 33, 14, 2883-2899 (2000)) on the aforementioned novel method for determination of hydrogen peroxide with the mimetic enzymes β-CD-hemin and hemin showed that β-CD-hemin was the best substrate for chlorophenol derivatives or phenol for hydrogen peroxide determination.

US 2010/0150857 A1 discloses a method for dyeing keratinous fibres. Example A3 discloses a two-part composition, the first part consisting of 5 g catechin, 5 g hexylene glycol, 3.75 g sodium lauryl ether sulphate, 0.036 g manganese chloride tetrahydrate, 1.2 g hydrogen peroxide, citric acid or sodium hydroxide to pH 5 and the remainder deionised water. The second part consists of 2.6 g sodium bicarbonate, 1 g carbomer, monoethanolamine to pH 9.0 and the remainder deionised water.

There remains a continuing need for improved hair colour compositions.

### SUMMARY OF THE INVENTION

In a first aspect of the invention, a hair colour composition is provided, the composition comprising a flavonoid, a catalyst comprising iron (II) or cobalt (II) complexed to a non-proteinaceous heterocyclic macrocycle ligand composed of pyrrole or pyrroline subunits, and either a hydrogen peroxide generator or 0.0001 to less than 1.5% w/w hydrogen peroxide; wherein the flavonoid is selected from the group consisting of flavones, isoflavones, flavans, isoflavans, flavanones, flavonols, flavan-3-ols, dihydroflavonol, flavanonols, proanthocyanidins, aurones, chalcones, dihydrochalcones, flavonolignans, and derivatives thereof, wherein the hair colour composition has a pH of 4.5 to 8.0, preferably 5.5 to 7.5.

In a second aspect of the invention, a kit for colouring hair is provided, the kit comprising:
(a) A hair composition according to any one of the preceding claims; and
(b) A pre- or post-treatment composition comprising a metal ion suitable for coordinating to the flavonoid or the product of the reaction of the flavonoid in the presence of the catalyst.

In a third aspect of the invention, a method for colouring hair is provided, the method comprising the step of treating hair with the hair colour composition of the first aspect of the invention.

### SUMMARY OF THE FIGURES

The invention is exemplified with reference to the following figures in which:

FIG. 1a shows ΔE values determined following successive treatments of catechin/haemin/H₂O₂ and controls;

FIG. 1b shows L* values determined following successive treatments of catechin/haemin/H₂O₂ and controls;

FIG. 1c shows a* values determined following successive treatments of catechin/haemin/H₂O₂ and controls;

FIG. 1d shows b* values determined following successive treatments of catechin/haemin/H₂O₂ and controls;

FIG. 2 shows ΔE values determined following successive treatments of catechin/haemin/H₂O₂ followed by sequential shampoo washes;

FIG. 3 shows ΔE values determined following a single treatment of luteolin or taxifolin with the combination of haemin/H₂O₂ followed by a single shampoo wash; and

FIG. 4 shows ΔE values determined following successive treatments of catechin/sodium iron chlorophyllin (FeChl)/H₂O₂ at pH 5.5 followed by sequential shampoo washes;

FIG. 5 shows ΔE values determined following successive treatments of catechin/sodium iron chlorophyllin (FeChl)/H₂O₂ at pH 7.5 followed by sequential shampoo washes;

FIG. 6 shows ΔE values determined following two treatments of catechin/sodium iron chlorophyllin (FeChl)/H₂O₂ at pH 5.5 or 8.0 followed by sequential shampoo washes;

FIG. 7 shows ΔE values determined following a single treatment of catechin/cobalt (II) or zinc (II) or copper (II) or tin (II) protoporphyrin (CoPP, ZNpp, CuPP and SnPP respectively) or protoporphyrin IX (PP)/H₂O₂ at pH 5.5 followed by a shampoo wash;

FIG. 8 shows ΔE values determined following two treatments of catechin/cobalt (II) protoporphyrin (CoPP)/H₂O₂ at pH 5.5 followed by sequential shampoo washes;

FIG. 9 shows ΔE values determined following a single treatment of catechin/sodium copper chlorophyllin (CuChl)/H₂O₂ at pH 5.5 followed by a shampoo wash;

FIG. 10 shows ΔE values determined following a single treatment of catechin/magnesium chlorophyll A (Chloro)/H₂O₂ at pH 5.5 followed by a shampoo wash; and

FIG. 11 shows ΔE values determined following a single treatment of catechin//H₂O₂ with either a two part manganese chloride tetrahydrate/sodium bicarbonate catalyst, the former at pH 5.5 and the latter at pH 9.0, or a single part haemin catalyst at pH 5.5 followed by a shampoo wash.

### DETAILED DESCRIPTION OF THE INVENTION

The inventors have provided a hair colour composition comprising a flavonoid, a catalyst comprising iron (II) or cobalt (II) complexed to a non-proteinaceous heterocyclic macrocycle ligand composed of pyrrole or pyrroline subunits, and either a hydrogen peroxide generator or 0.0001 to less than 1.5% w/w hydrogen peroxide; wherein the flavonoid is selected from the group consisting of flavones, isoflavones, flavans, isoflavans, flavanones, flavonols, flavan-3-ols, dihydroflavonol, flavanonols, proanthocyanidins, aurones, chalcones, dihydrochalcones, flavonolignans, and derivatives thereof, wherein the hair colour composition has a pH of 4.5 to 8.0, preferably 5.5 to 7.5. By derivatives are meant, for example, glycosides or esters.

The flavone may be selected from the group consisting of apigenin, luteolin and chrysin; the isoflavone may be selected from the group consisting of daidzein, genistein, formononetin and orobol; the flavan may be 4′-hydroxy-5,6-dimethoxyflavan; the isoflavan may be glabridin or licoricidin; the flavanone may be eriodictyol or naringenin; the flavonol may be selected from the group consisting of myricetin, kaempferol, quercetin and gossypetin; the flavan-3-ol may be selected from the group consisting of catechin, theaflavin, gallocatechin, catechin gallate, gallocatechin gallate, epicatechin, epigallocatechin, epigallocatechin gallate and epigallocatechin gallate; the dihydroflavonol may be taxifolin or aromadendrin; the aurone may be sulphuretin; the chalcone may be 2′-hydroxy-4-methoxy-chalcone; the dihydrochalcone may be phloretin or phloridzin; and the flavonolignan may be silibinin or silichristin.

The non-proteinaceous heterocyclic macrocycle ligand composed of pyrrole or pyrroline subunits may be selected from the group consisting of protoporphyrins, corrins, porphyrins, porphorinogens, chlorins bacteriochlorins, isobacteriochlorins, corphins and corrole. Thus the catalyst may be selected from the group consisting of cobalamins, haemin, Haem A, Haem B, Haem C, Haem O, iron (II) chlorophyllin and siroheme.

Preferably the catalyst is selected from the group consisting of haemin, sodium iron chlorophyllin and cobalt (II) protoporphyrin.

The hair composition may comprise 0.001-10, preferably 0.01-5, most preferably 0.1-2% w/w flavonoid; 0.001-1, preferably 0.01-0.5% w/w hydrogen peroxide; and 0.0001-5, preferably 0.001-1% w/w catalyst.

As an alternative to hydrogen peroxide, the hydrogen peroxide may be generated in-situ by a hydrogen peroxide generator hereby reducing the inconvenience, e.g. allergenic, of having a higher concentration in the inventive composition when first applied to the hair. Thus the hydrogen peroxide generator may comprise a hydrogen peroxide generating oxidase, a substrate and oxygen. The hydrogen peroxide generating oxidase may be selected from the group consisting of (S)-2-hydroxy acid oxidase, D-galactose oxidase, glucose oxidase, coniferyl alcohol oxidase, glycolate oxidase, hexose oxidase, oxalate oxidase, amino acid oxidase and L-galactonolactone oxidase and the respective substrate is selected from the group consisting of (S)-2-hydroxy acid, D-galactose, glucose, coniferyl alcohol, α-hydroxy acids, D-glucose, oxalic acid, (S)-lactate, L-galactono-1,4-lactone and amino acid. Thus the hydrogen peroxide generator may be selected from the group consisting of (S)-2-hydroxy acid with (S)-2-hydroxy acid oxidase, D-galactose with D-galactose oxidase, glucose with glucose oxidase, coniferyl alcohol with coniferyl alcohol oxidase, α-hydroxy acids with glycolate oxidase, D-glucose with hexose oxidase, oxalic acid with oxalate oxidase, (S)-lactate with lactate oxidase, L-galactono-1,4-lactone with L-galactonolactone oxidase, and amino acid with amino acid oxidase, all in the presence of oxygen.

The hair composition may comprise 0.0001-5, preferably 0.001-1% w/w hydrogen peroxide generating oxidase and 0.01-10, preferably 0.1-5% w/w substrate.

The pH of the hair composition may be pH of 4.0 to 8.5, preferably 4.5 to 8.0.

In one embodiment, the flavonoid is colourless.

Preferred formats for the hair composition are a shampoo and/or a conditioner.

The inventors also provide a kit for colouring hair comprising:
(a) A hair composition according to the invention; and
(b) A pre- or post-treatment composition comprising a metal ion suitable for coordinating to the flavonoid or the product of the reaction of the flavonoid in the presence of the catalyst.

Use of the aforementioned metal ion modulates the colour provided by the flavonoid, hydrogen peroxide and catalyst.

The metal ion may be selected from the group consisting of iron (II), iron (III), cobalt (II), copper (I), copper (II), copper (III), aluminium (III), zinc (I), zinc (II), manganese (II), manganese (III), manganese (IV), manganese (V), manganese (VI) and manganese (VII). The pre- or post-treatment composition may comprise 0.001-10, preferably 0.01-5, more preferably 0.01-2% w/w metal ion, and further may have a pH of 4.0 to 8.5, preferably 4.5 to 8.0. The pre- or post-treatment composition may also be a shampoo and/or a conditioner.

The inventors also provide a method for colouring hair comprising the step of treating hair with the hair colour composition of the invention. In one embodiment, the method comprises the additional steps of:

(a) Treating the hair with the aforesaid pre- or post-treatment composition; and
(b) Washing the hair;
wherein step (a) is completed prior to or after treatment of the hair with the hair composition; and
wherein step (b) is completed between step (a) and treatment of the hair with the hair composition.

By including an intermediate washing step between application of the metal ion composition and the hair colour composition, staining of the scalp is reduced.

### 1.0 Shampoo Compositions

Shampoo compositions will nearly always comprise a cleansing surfactant component in an aqueous base.

### 1.1 Cleansing Surfactant

The cleansing surfactant may consist of a single surfactant, usually an anionic surfactant (to provide foam) such as sodium lauryl ether sulphate, or more commonly a mixture of sodium lauryl ether sulphate with a co-surfactant to provide mildness. The most preferred co-surfactant is cocoamidopropyl betaine.

The total amount of surfactant (including any co-surfactant, and/or any emulsifier) in a shampoo composition may be from 1 to 50, preferably from 2 to 40, more preferably from 10 to 25% w/w. Compositions comprising more than 25% w/w cleansing surfactant are commonly considered concentrated shampoos.

Examples of suitable anionic cleansing surfactants are the alkyl sulphates, alkyl ether sulphates, alkaryl sulphonates, alkanoyl isethionates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium and mono-, di- and triethanolamine salts. The alkyl and acyl groups generally contain from 8 to 18, preferably from 10 to 16 carbon atoms and may be unsaturated. The alkyl ether sulphates, alkyl ether sulphosuccinates, alkyl ether phosphates and alkyl ether carboxylic acids and salts thereof may contain from 1 to 20 ethylene oxide or propylene oxide units per molecule.

Typical anionic cleansing surfactants for use in shampoo compositions of the invention include sodium oleyl succinate, ammonium lauryl sulphosuccinate, sodium lauryl sulphate, sodium lauryl ether sulphate, sodium lauryl ether sulphosuccinate, ammonium lauryl sulphate, ammonium lauryl ether sulphate, sodium dodecylbenzene sulphonate, triethanolamine dodecylbenzene sulphonate, sodium cocoyl isethionate, sodium lauryl isethionate, lauryl ether carboxylic acid and sodium N-lauryl sarcosinate.

Preferred anionic surfactants are the alkyl sulfates and alkyl ether sulfates. These materials have the respective formulae R2OSO₃M and R1O(C₂H₄O)ₓSO₃M, wherein R2 is alkyl or alkenyl of from 8 to 18 carbon atoms, x is an integer having a value of from about 1 to about 10, and M is a cation such as ammonium, alkanolamines, such as triethanolamine, monovalent metals, such as sodium and potassium, and polyvalent metal cations, such as magnesium, and calcium. Most preferably R2 has 12 to 14 carbon atoms, in a linear rather than branched chain.

Preferred anionic cleansing surfactants are selected from sodium lauryl sulphate and sodium lauryl ether sulphate(n)EO, (where n is from 1 to 3); more preferably sodium lauryl ether sulphate(n)EO, (where n is from 1 to 3); most preferably sodium lauryl ether sulphate(n)EO where n=1.

Preferably the level of alkyl ether sulphate is from 0.5 to 25, more preferably from 3 to 18, most preferably from 6 to 15% w/w of the composition.

The total amount of anionic cleansing surfactant in shampoo compositions of the invention generally ranges from 0.5 to 45, more preferably from 1.5 to 20% w/w of the composition.

### 1.2 Nonionic Surfactant

Shampoo compositions of the invention may contain non-ionic surfactant. Most preferably non-ionic surfactants are present in the range 0 to 5% w/w of the composition.

Nonionic surfactants that can be included in shampoo compositions of the invention include condensation products of aliphatic (C8-C18) primary or secondary linear or branched chain alcohols or phenols with alkylene oxides, usually ethylene oxide and generally having from 6 to 30 ethylene oxide groups. Alkyl ethoxylates are particularly preferred. Most preferred are alkyl ethoxylates having the formula R—(OCH₂CH₂)nOH, where R is an alkyl chain of C12 to C15, and n is 5 to 9.

Other suitable nonionic surfactants include mono- or di-alkyl alkanolamides. Examples include coco mono- or di-ethanolamide and coco mono-isopropanolamide.

Further nonionic surfactants which can be included in shampoo compositions of the invention are the alkyl polyglycosides (APGs). Typically, APG is one which comprises an alkyl group connected (optionally via a bridging group) to a block of one or more glycosyl groups. Preferred APGs are defined by the following formula RO-(G)ₙ wherein R is a branched or straight chain alkyl group which may be saturated or unsaturated and G is a saccharide group.

R may represent a mean alkyl chain length of from about C5 to about C20. Preferably R represents a mean alkyl chain length of from about C8 to about C12. Most preferably the value of R lies between about 9.5 and about 10.5. G may be selected from C5 or C6 monosaccharide residues, and is preferably a glucoside. G may be selected from the group comprising glucose, xylose, lactose, fructose, mannose and derivatives thereof. Preferably G is glucose. The degree of polymerisation, n, may have a value of from about 1 to about 10 or more, preferably a value of from about 1.1 to about 2, most preferably a value of from about 1.3 to about 1.5. Suitable alkyl polyglycosides for use in the invention are commercially available and include for example those materials identified as: Oramix NS10 ex Seppic; Plantaren 1200 and Plantaren 2000 ex Henkel.

Other sugar-derived nonionic surfactants which can be included in compositions of the invention include the C10-C18 N-alkyl (Cl-C6) polyhydroxy fatty acid amides, such as the C12-C18 N-methyl glucamides, as described for example in WO 92/06154 and U.S. Pat. No. 5,194,639, and the N-alkoxy polyhydroxy fatty acid amides, such as C10-C18 N-(3-methoxypropyl) glucamide.

### 1.3 Amphoteric/Zwitterionic Surfactant

Amphoteric or zwitterionic surfactant can be included in an amount ranging from 0.5 to about 8, preferably from 1 to 4% w/w of the shampoo compositions of the invention.

Examples of amphoteric or zwitterionic surfactants include alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine, lauryl betaine, cocamidopropyl betaine and sodium cocoamphoacetate.

A particularly preferred amphoteric or zwitterionic surfactant is cocamidopropyl betaine.

Mixtures of any of the foregoing amphoteric or zwitterionic surfactants may also be suitable. Preferred mixtures are those of cocamidopropyl betaine with further amphoteric or zwitterionic surfactants as described above. A preferred further amphoteric or zwitterionic surfactant is sodium cocoamphoacetate.

### 1.4 Suspending Agents

Preferably an aqueous shampoo composition of the invention further comprises a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. Carbopol 980 is the commonly used suspending agent though there is a growing desire to find an alternative. All Carbopol (trademark) materials are available from Goodrich.

Suitable cross-linked polymers of acrylic acid and acrylate esters are Pemulen TR1 or Pemulen TR2. A suitable heteropolysaccharide gum is xanthan gum, for example that available as Kelzan mu.

Mixtures of any of the above suspending agents may be used. Preferred is a mixture of cross-linked polymer of acrylic acid and crystalline long chain acyl derivative.

Suspending agent will generally be present in a shampoo composition of the invention at levels of from 0.1 to 10, preferably from 0.5 to 6, more preferably from 0.9 to 4% w/w of the composition. Generally such suspending agents are present at around 2% w/w of the composition.

### 1.5 Water

Shampoo compositions of the invention are generally aqueous, i.e. they have water or an aqueous solution or a lyotropic liquid crystalline phase as their major component. Suitably, the composition will comprise from 50 to 98, preferably from 60 to 90% w/w of the composition.

Typically, shampoo compositions have a pH of around 5.5.

### 1.6 Optional Ingredients

The shampoo compositions of the invention might also contain the following optional ingredients: conditioning agents;

### 1.6.1 Conditioning Agents

Conditioning actives are often included in shampoo compositions. These are sometimes called ‘2-in-1’ formulations. Conditioning actives fall into three classes:
silicones (and cationic deposition polymers to assist in silicone deposition)
cationic surfactants
non-silicone oils

Where silicones are included, the composition is likely to also contain a cationic deposition polymer for enhancing deposition of the silicone. Further, a silicone-containing composition is likely to be lamellar as opposed to isotropic. Isotropic compositions do not deposit silicone effectively.

### 1.6.1.1 Silicones

The shampoo compositions of the invention can contain emulsified droplets of a silicone conditioning agent, for enhancing conditioning performance.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

Examples of suitable pre-formed emulsions include Xiameter MEM 1785 and microemulsion DC2-1865 available from Dow Corning. These are emulsions/microemulsions of dimethiconol. Cross-linked silicone gums are also available in a pre-emulsified form, which is advantageous for ease of formulation.

A further preferred class of silicones for inclusion in shampoos and conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”.

Specific examples of amino functional silicones suitable for use in the invention are the aminosilicone oils DC2-8220, DC2-8166 and DC2-8566 (all ex Dow Corning).

The most commonly used amino silicone is sourced from Dow Corning and is coded DC7134. Pre-formed emulsions of amino functional silicone are also available from suppliers of silicone oils such as Dow Corning and General Electric. Specific examples include DC939 Cationic Emulsion and the non-ionic emulsions DC2-7224, DC2-8467, DC2-8177 and DC2-8154 (all ex Dow Corning).

Suitable quaternary silicone polymers are described in EP-A-0 530 974. A preferred quaternary silicone polymer is K3474, ex Goldschmidt.

With some shampoos it is preferred to use a combination of amino and non amino functional silicones.

Emulsified silicones for use in the shampoo compositions of the invention will typically have an average silicone droplet size in the composition of less than 30, preferably less than 20, more preferably less than 10 micron, ideally from 0.01 to 1 micron. Silicone emulsions having an average silicone droplet size of about 0.15 micron are generally termed microemulsions.

Emulsified silicones for use in the conditioner compositions of the invention will typically have a size in the composition of less than 30, preferably less than 20, more preferably less than 15. Preferably the average silicone droplet is greater than 0.5 micron, more preferably greater than 1 micron, ideally from 2 to 8 micron.

Silicone particle size may be measured by means of a laser light scattering technique, for example using a 2600D Particle Sizer from Malvern Instruments.

The viscosity of the emulsified silicone itself (not the emulsion or the final hair conditioning composition) is typically at least 10,000, preferably at least 60,000, most preferably at least 500,000, ideally at least 1,000,000 cst at 25° C. Preferably the viscosity does not exceed 10⁹ cst at 25° C. for ease of formulation.

The total amount of silicone is preferably from 0.01 to 10, more preferably from 0.1 to 5, most preferably 0.5 to 3% w/w of the composition of the invention.

Also suitable are emulsions of amino functional silicone oils with non ionic and/or cationic surfactant.

Cationic deposition polymers are used to deposit the silicone droplets to the hair surface and hence enhance performance.

Suitable cationic polymers may be homopolymers which are cationically substituted or may be formed from two or more types of monomers. The weight average (Mw) molecular weight of the polymers will generally be between 100 000 and 2 million daltons. The polymers will have cationic nitrogen containing groups such as quaternary ammonium or protonated amino groups, or a mixture thereof. If the molecular weight of the polymer is too low, then the conditioning effect is poor. If too high, then there may be problems of high extensional viscosity leading to stringiness of the composition when it is poured.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory. The ratio of the cationic to non-cationic monomer units is selected to give polymers having a cationic charge density in the required range, which is generally from 0.2 to 3.0 meq/gm. The cationic charge density of the polymer is suitably determined via the Kjeldahl method as described in the US Pharmacopoeia under chemical tests for nitrogen determination.

Suitable cationic polymers include, for example, copolymers of vinyl monomers having cationic amine or quaternary ammonium functionalities with water soluble spacer monomers such as (meth)acrylamide, alkyl and dialkyl(meth)acrylamides, alkyl(meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerized in the amine form and then converted to ammonium by quaternization.

The cationic polymers can comprise mixtures of monomer units derived from amine- and/or quaternary ammonium-substituted monomer and/or compatible spacer monomers.

Suitable cationic polymers include, for example:
cationic diallyl quaternary ammonium-containing polymers including, for example, dimethyldiallylammonium chloride homopolymer and copolymers of acrylamide and dimethyldiallylammonium chloride, referred to in the industry (CTFA) as Polyquaternium 6 and Polyquaternium 7, respectively;
mineral acid salts of amino-alkyl esters of homo- and co-polymers of unsaturated carboxylic acids having from 3 to 5 carbon atoms, (as described in U.S. Pat. No. 4,009,256); and
cationic polyacrylamides (as described in WO95/22311).

Other cationic polymers that can be used include cationic polysaccharide polymers, such as cationic cellulose derivatives, cationic starch derivatives, and cationic guar gum derivatives.

Cationic polysaccharide polymers suitable for use in compositions of the invention include monomers of the formula A-O—[R—N+(R1)(R2)(R3)X-] wherein A is an anhydroglucose residual group, such as a starch or cellulose anhydroglucose residual; R is an alkylene, oxyalkylene, polyoxyalkylene, or hydroxyalkylene group, or combination thereof; R1, R2 and R3 independently represent alkyl, aryl, alkylaryl, arylalkyl, alkoxyalkyl, or alkoxyaryl groups, each group containing up to about 18 carbon atoms; the total number of carbon atoms for each cationic moiety (i.e., the sum of carbon atoms in R1, R2 and R3) is preferably about 20 or less; and X is an anionic counterion.

Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from the Amerchol Corporation, for instance under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimethylammonium chloride (commercially available from Rhodia in their JAGUAR trademark series). Examples of such materials are JAGUAR C13S, JAGUAR C14, JAGUAR C15, JAGUAR C17 and JAGUAR C16 Jaguar CHT and JAGUAR C162.

Mixtures of any of the above cationic polymers may be used.

Cationic polymer will generally be present in a shampoo composition of the invention at levels of from 0.01 to 5, preferably from 0.05 to 1, more preferably from 0.08 to 0.5% w/w of the weight of the compositions of the invention.

### 1.6.1.2 Cationic Surfactants

Cationic surfactants may be used in 2-in-1 shampoos to provide a conditioning benefit. However, since a shampoo composition is likely to also comprise anionic cleansing surfactants, the use of cationic surfactants is limited to compositions where the cationic surfactant is separated from the anionic phase by way of a stable conditioning gel phase made separately from the rest of the formulation and then incorporated afterwards.

### 1.6.1.3 Non-Silicone Oils

These are typically hydrocarbon oils or fatty alcohols. A fatty alcohol is nearly always included in a conditioning composition and often included in 2-in-1 shampoos. Cetearyl alcohol is one of the preferred examples.

### 1.6.2 Fibre Actives

Fibre actives are provided to repair or coat the hair fibres. Examples are trehalose (a disaccharide), adipic acid (dicarboxylic acid) and gluconolactone.

### 1.6.3 Anti-Dandruff Actives

There are two classes of anti-dandruff active: the azoles and the pyrithiones, both are active against the target fungi malassezia spp. The azoles include ketoconazole and climbazole which are fat soluble actives. The pyrithiones include zinc pyrithione (ZPT) which is insoluble and delivered as a particle to the scalp.

Preferably, the antidandruff active is present at from 0.01 to 5, more preferably from 0.1 to 2.5% w/w of the composition of the invention.

### 2.0 Hair Conditioning Compositions

The compositions of the invention may also be hair conditioning compositions (also known as conditioners). A conditioner which is to be used after a shampoo is known as a ‘system conditioner’ whereas one which is included in a shampoo composition is known as a ‘2-in-1’. Hair conditioning compositions may also be left on the head, i.e. not rinsed off after application. These are known as Leave-on-Treatments (LOTs) as opposed to Rinse-off-Treatments (ROTs).

The main ingredients in a system conditioner are the conditioning actives described above, the main actives being a cationic surfactant (e.g. behenyltrimmonium chloride), a silicone conditioning agent (e.g. aminosilicone (DC 7134)) and a non-silicone oil, usually a fatty alcohol (e.g. cetearyl alcohol).

Anti-dandruff actives may also be included in hair conditioning compositions of the invention.

### 2.1 Cationic Surfactants

Preferably, the cationic surfactants have the formula N+R1R2R3R4 wherein R1, R2, R3 and R4 are independently (C1 to C30) alkyl or benzyl. Preferably, one, two or three of R1, R2, R3 and R4 are independently (C4 to C30) alkyl and the other R1, R2, R3 and R4 group or groups are (C1-C6) alkyl or benzyl. More preferably, one or two of R1, R2, R3 and R4 are independently (C6 to C30) alkyl and the other R1, R2, R3 and R4 groups are (C1-C6) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (e.g. oleyl). Alkyl groups are optionally ethoxylated on the alkyl chain with one or more ethyleneoxy groups.

Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN KDMP, ex Clariant.

Another example of a class of suitable cationic surfactants for use in the invention, either alone or together with one or more other cationic surfactants, is a combination of (i) and (ii) below:

(i) an amidoamine corresponding to the general formula (I) R1CONH(CH₂)mN(R2)R3
in which R1 is a hydrocarbyl chain having 10 or more carbon atoms, R2 and R3 are independently selected from hydrocarbyl chains of from 1 to 10 carbon atoms, and m is an integer from 1 to about 10; and
   (ii) an acid.

As used herein, the term hydrocarbyl chain means an alkyl or alkenyl chain. Preferred amidoamine compounds are those corresponding to formula (I) in which R1 is a hydrocarbyl residue having from about 11 to about 24 carbon atoms, R2 and R3 are each independently hydrocarbyl residues, preferably alkyl groups, having from 1 to about 4 carbon atoms, and m is an integer from 1 to about 4. Preferably R2 and R3 are methyl or ethyl groups. Preferably m is 2 or 3, i.e. an ethylene or propylene group.

Preferred amidoamines useful herein include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof. Particularly preferred amidoamines useful herein are stearamidopropyldimethylamine, stearamidoethyldiethylamine, and mixtures thereof.

Commercially available amidoamines useful herein include: stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

The acid may be any organic or mineral acid which is capable of protonating the amidoamine in the conditioner composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, lactic acid and mixtures thereof.

The primary role of the acid is to protonate the amidoamine in the hair treatment composition thus forming a tertiary amine salt (TAS) in-situ in the hair treatment composition. The TAS in effect is a non-permanent quaternary ammonium or pseudo-quaternary ammonium cationic surfactant. Suitably, the acid is included in a sufficient amount to protonate more than 95 mole % (20° C.) of the amidoamine present.

In conditioners of the invention, the level of cationic surfactant will generally range from 0.01 to 10, more preferably 0.05 to 7.5, most preferably 0.1 to 5% by weight of the composition.

### 2.2 Silicone Conditioning Agent

The compositions of the invention can contain emulsified droplets of a silicone conditioning agent, for enhancing conditioning performance as previously described.

### 2.3 Non-Silicone Oils

Compositions according to the present invention may also comprise a dispersed, non-volatile, water-insoluble, non-silicone oily conditioning agent. Preferably such non-silicone oily conditioning agents are present in the hair conditioning compositions of the invention. By “insoluble” is meant that the material is not soluble in water (distilled or equivalent) at a concentration of 0.1% w/w at 25° C. Suitable non-silicone oils are selected from hydrocarbon oils, fatty esters and mixtures thereof.

Straight chain hydrocarbon oils will preferably contain from about 12 to about 30 carbon atoms. Also suitable are polymeric hydrocarbons of alkenyl monomers, such as C2-C6 alkenyl monomers. Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used.

Suitable fatty esters are characterised by having at least 10 carbon atoms, and include esters with hydrocarbyl chains derived from fatty acids or alcohols, Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used. Particularly preferred fatty esters are mono-, di- and triglycerides, more specifically the mono-, di-, and tri-esters of glycerol and long chain carboxylic acids such as C1-C22 carboxylic acids. Preferred materials include cocoa butter, palm stearin, sunflower oil, soyabean oil and coconut oil.

The oily or fatty material is suitably present at a level of from 0.05 to 10, preferably from 0.2 to 5, more preferably from about 0.5 to 3% w/w of the composition of the invention.

### 2.4 Fatty Alcohols

Hair conditioning compositions of the invention will typically also incorporate a fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol in conditioners of the invention will generally range from 0.01 to 10, preferably from 0.1 to 8, more preferably from 0.2 to 7, most preferably from 0.3 to 6% w/w by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

### 3.0 Examples

### Example 1

### Materials

3% w/w aqueous H₂O₂ (Sigma, UK)

(+)-catechin (Sigma, UK)

Haemin (Sigma, UK)

Britton Robinson buffer ingredients (boric acid, phosphoric acid and glacial acetic acid) (Sigma, UK)

NaOH (Sigma, UK)

7 g/25 cm Natural white hair switches (International Hair Importers, USA)

### Method

Small (0.5 g/5 cm) hair switches were prepared from the 7 g/25 cm switches and used for evaluating colour uptake. Prior to any experimental treatments, hair switches were soaked in 10% sodium dodecyl sulphate for 1 hour, rinsed in water, soaked for a further minute in 1% sodium dodecyl sulphate, rinsed in water again and then shampoo washed and finally dried using a hair dryer for ˜1 minute. Baseline L*a*b* readings were determined using a Minolta spectrophotometer (CM508d Minolta, UK) for all switches. Hair switches were then incubated in the reactions set forth in table 1 at room temperature for 30 minutes. Following incubation, the hair switches were rinsed in running water, washed in shampoo and dried using a hair dryer for ˜1 minute. Colour generation following treatment was evaluated by determining L*a*b* readings using the Minolta spectrophotometer. Hair switches were treated for a further 4 applications of the respective reactions by repeating the foregoing colouring, rinsing, washing and drying steps. Increase in colour uptake after each application was determined from the L*a*b* readings by calculating the ΔE values according to the equation below:

ΔE=√(L*_{B}−L*_{D})²+(a*_{B}−a*_{D})²+(b*_{B}−b*_{D})²

where

B=blank/control (undyed/untreated)
D=dyed/treated
L*=lightness (where 0=black and 100=diffuse white)
a*=green/red (negative values indicate green and positive values indicate red)
b*=blue/yellow (negative values indicate blue and positive values indicate yellow)

In order to assess substantivity of colour, the hair switches were shampoo washed 5 times. L*a*b* readings were determined after each wash.

TABLE 1
Hair colour compositions. Total volume in all reactions was 6000 μl and
therefore the initial concentrations for each of the ingredients (where present)
was 17.6 mM catechin, 46 μM haemin and 0.3% w/w hydrogen peroxide.

| | Volume | Volume | Volume | Volume | Volume | Volume | Volume |
|---|---|---|---|---|---|---|---|
| Ingredient | (μl) | (μl) | (μl) | (μl) | (μl) | (μl) | (μl) |
| Catechin | 600 | 600 | 0 | 0 | 0 | 0 | 600 |
| Haemin | 600 | 0 | 0 | 600 | 0 | 600 | 600 |
| H₂O₂ (3% | 600 | 0 | 0 | 0 | 600 | 600 | 0 |
| w/w) | | | | | | | |
| 50 mM Britton | 4200 | 5400 | 6000 | 5400 | 5400 | 4800 | 4800 |
| Robinson | | | | | | | |
| buffer pH 5.5 | | | | | | | |

### Results

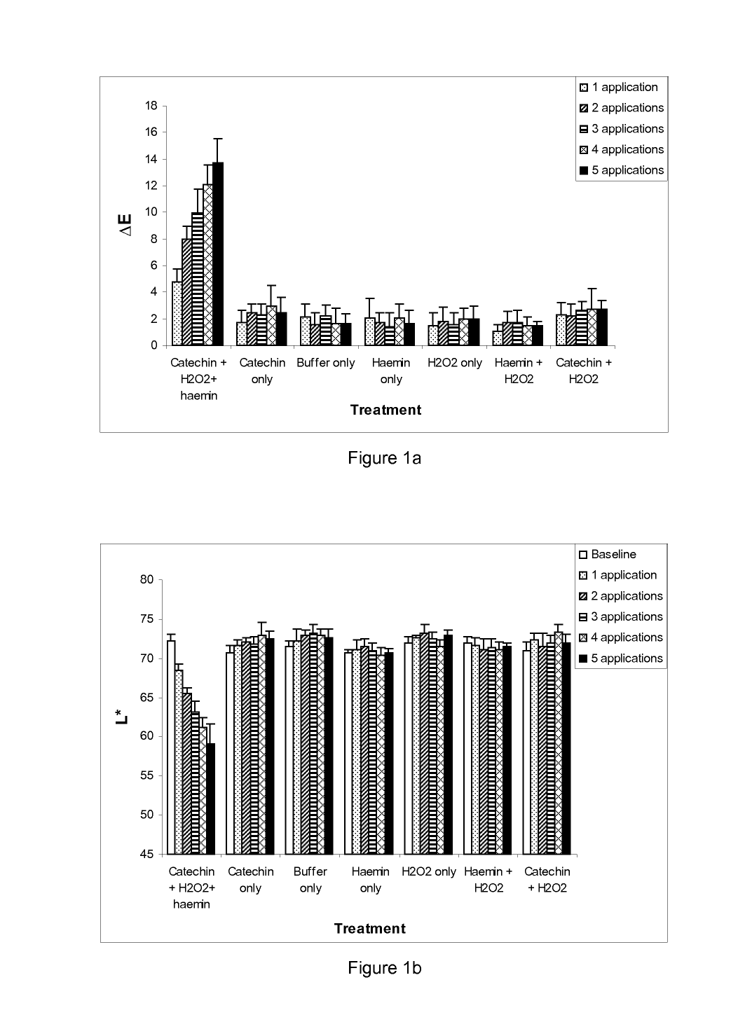

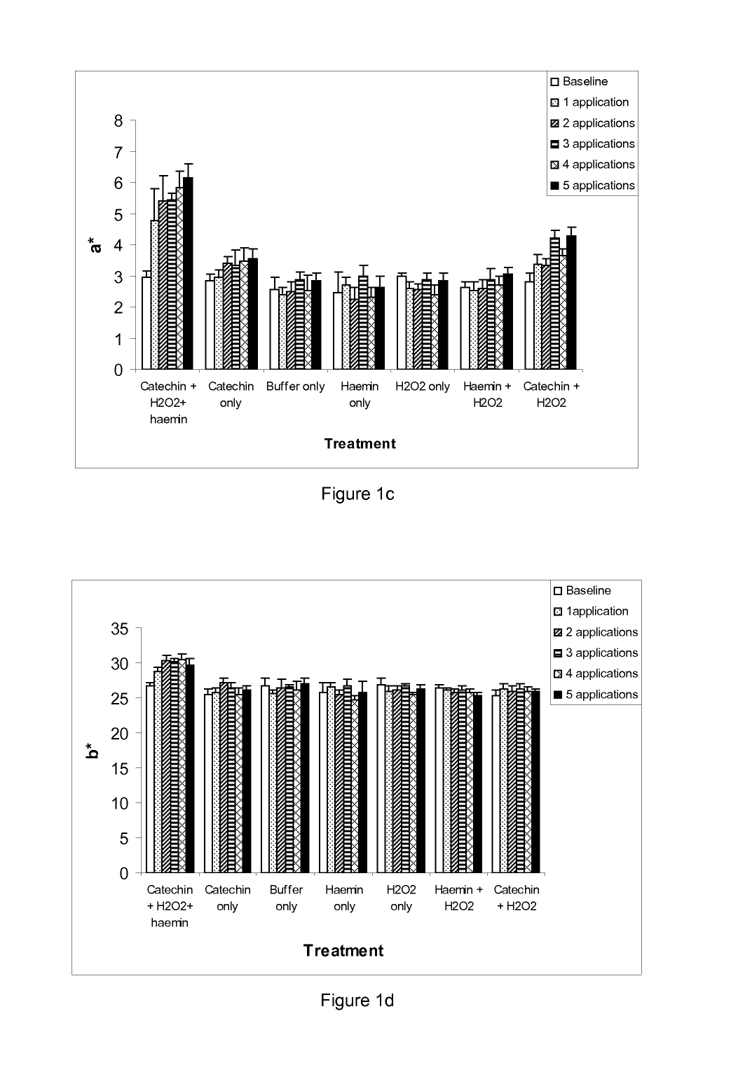

Readings for L*a*b* were used to calculate ΔE after each application. The results for each of the reactions in table 1 are shown in FIG. 1a. In each case, buffer is present. Each bar is the mean of 6 readings per hair switch from a single representative experiment. A progressive increase in ΔE was observed following each treatment of catechin/haemin/H₂O₂ up to a maximum average ΔE of 13.76.

The mean L*, a* and b* readings of 6 readings per hair switch for each of the reactions in table 1 from a single representative experiment are illustrated in FIGS. 1b, 1c and 1d respectively. In each case, buffer is present. A decrease in L* was observed following each treatment of catechin/haemin/H₂O₂ suggesting that the shade of the hair was becoming darker compared to controls (see FIG. 1b). An increase in a* was also observed following each treatment of catechin/haemin/H₂O₂ suggesting that the shade of the hair was becoming more red compared to controls (see FIG. 1c). A slight increase in b* values was observed after 1 and 2 applications of catechin/haemin/H₂O₂ suggesting the shade was becoming more yellow (see FIG. 1d).

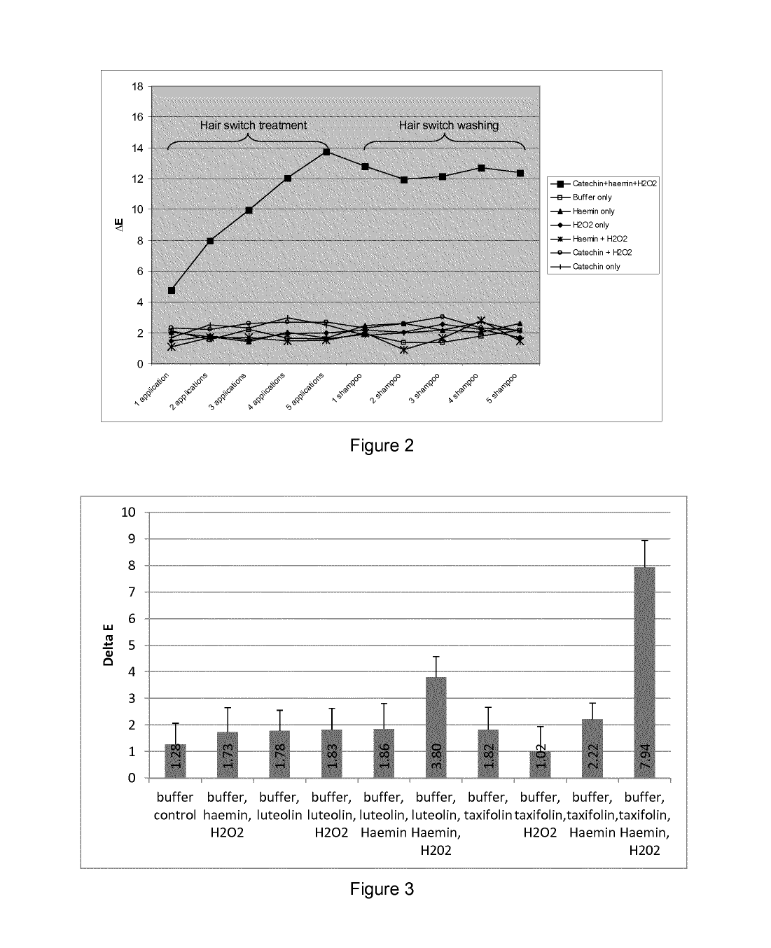

Readings for L*a*b* were also used to calculate ΔE following sequential shampoo washes to determine substantivity of the colour in the hair fibres. The results for each reaction in table 1 are illustrated in FIG. 2 where each value represents the mean of 6 readings per hair switch from a single representative experiment. In each case, buffer is present. A small reduction in ΔE was found in catechin/haemin/H₂O₂ treated switches sequentially washed 5× with shampoo suggesting the colour is being retained within the hair fibre.

### Example 2

### Materials

Luteolin (Sigma Aldrich)

Taxifolin (Sigma Aldrich)

### Method

The method was identical to that set forth in Example 1 except that the hair switches underwent only a single treatment with the hair colour compositions. The hair colour compositions are set forth in tables 2 and 3.

TABLE 2
Hair colour compositions. Total volume in all reactions was
6000 μl and therefore the initial concentrations for each
of the ingredients (where present) was 0.67% luteolin,
460 μM haemin and 0.3% w/w hydrogen peroxide.

| | Volume | Volume | Volume | Volume | Volume | Volume |
|---|---|---|---|---|---|---|
| Ingredient | (μl) | (μl) | (μl) | (μl) | (μl) | (μl) |
| Luteolin | 100 | 100 | 0 | 100 | 0 | 100 |
| (40 mg/ml) | | | | | | |
| Haemin | 600 | 0 | 0 | 0 | 600 | 600 |
| H₂O₂ (3% w/w) | 600 | 0 | 0 | 600 | 600 | 0 |
| 50 mM Britton | 4700 | 5900 | 6000 | 5300 | 4800 | 5300 |
| Robinson | | | | | | |
| buffer pH 5.5 | | | | | | |

TABLE 3
Hair colour compositions. Total volume in all reactions was
6000 μl and therefore the initial concentrations for each
of the ingredients (where present) was 0.83% taxifolin,
460 μM haemin and 0.3% w/w hydrogen peroxide.

| | Volume | Volume | Volume | Volume | Volume | Volume |
|---|---|---|---|---|---|---|
| Ingredient | (μl) | (μl) | (μl) | (μl) | (μl) | (μl) |
| Taxifolin | 100 | 100 | 0 | 100 | 0 | 100 |
| (50 mg/ml) | | | | | | |
| Haemin | 600 | 0 | 0 | 0 | 600 | 600 |
| H₂O₂(3% w/w) | 600 | 0 | 0 | 600 | 600 | 0 |
| 50 mM Britton | 4700 | 5900 | 6000 | 5300 | 4800 | 5300 |
| Robinson | | | | | | |
| buffer pH 5.5 | | | | | | |

### Results

The results are presented in FIG. 3 and show that in the presence of haemin and hydrogen peroxide, a peroxidase-like oxidative reaction occurs that dyes hair to achieve more of a colour change than the relevant controls.

### Example 3

### Materials

Sodium iron chlorophyllin (inner Natural Ingredients Incorporated, China)

### Method

The method was identical to that set forth in Example 1 except only four rather than five treatments with the hair colour composition were performed. The hair colour compositions are set forth in table 4.

TABLE 4
Hair colour compositions. Total volume in all reactions was 6000 μl and
therefore the initial concentrations for each of the ingredients (where present) was
17.6 mM catechin, 0.03% iron chlorophyllin and 0.3% w/w hydrogen peroxide.

| | Volume | Volume | Volume | Volume | Volume | Volume | Volume | Volume |
|---|---|---|---|---|---|---|---|---|
| Ingredient | (μl) | (μl) | (μl) | (μl) | (μl) | (μl) | (μl) | (μl) |
| Catechin | 600 | 600 | 0 | 0 | 0 | 600 | 600 | 0 |
| (40 mg/ml) | | | | | | | | |
| Sodium | 600 | 0 | 0 | 0 | 600 | 600 | 0 | 600 |
| iron | | | | | | | | |
| chlorophyllin | | | | | | | | |
| (0.3% | | | | | | | | |
| w/w) | | | | | | | | |
| H₂O₂ (3% | 600 | 0 | 0 | 600 | 600 | 0 | 600 | 0 |
| w/w) | | | | | | | | |
| 50 mM | 4200 | 5400 | 6000 | 5300 | 4800 | 4800 | 4800 | 5400 |
| Britton | | | | | | | | |
| Robinson | | | | | | | | |
| buffer pH | | | | | | | | |
| 5.5 | | | | | | | | |

### Results

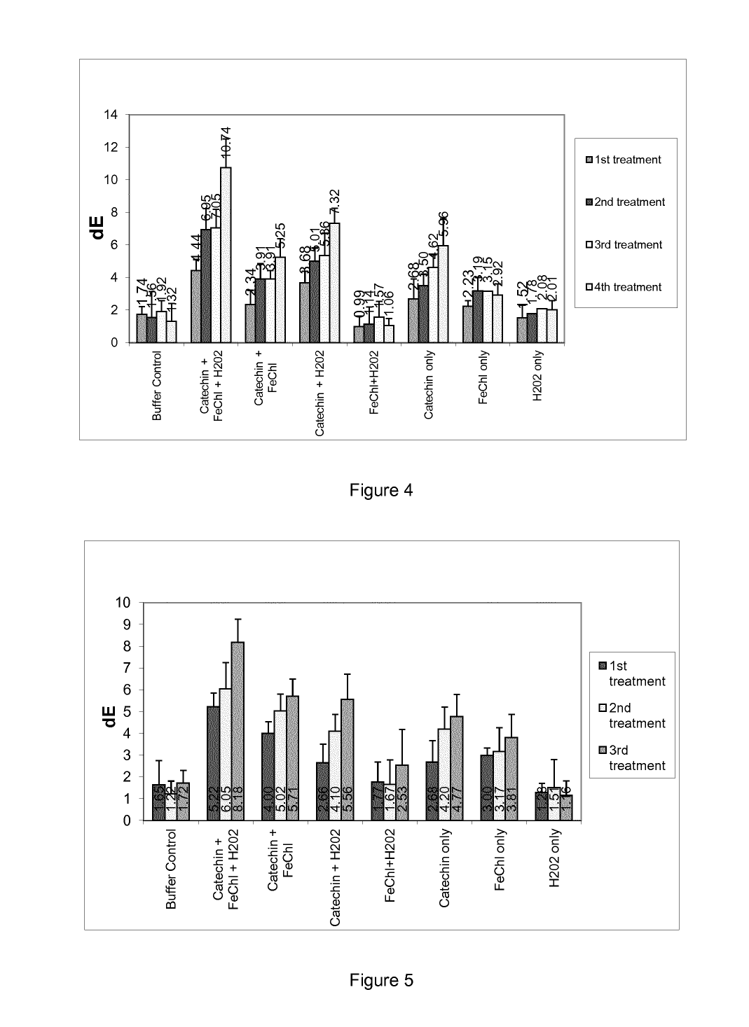

The results are presented in FIG. 4 and show that in the presence of catechin and hydrogen peroxide, a peroxidase-like oxidative reaction occurs that dyes hair to achieve more of a colour change than the relevant controls.

### Example 4

### Materials

pH 7.5 tris-HCl buffer (Sigma Aldrich)

### Method

Example 3 was repeated with a pH 7.5 tris-HCl buffer and for three rather than four treatments.

### Results

The results are presented in FIG. 5 and show slightly reduced colour change compared to the results for Example 3.

### Example 5

### Materials

Britton Robinson buffer pH 8.0 (Sigma Aldrich)

### Method

Example 3 was repeated at pH 5.5 and 8.0, both with two treatments only.

### Results

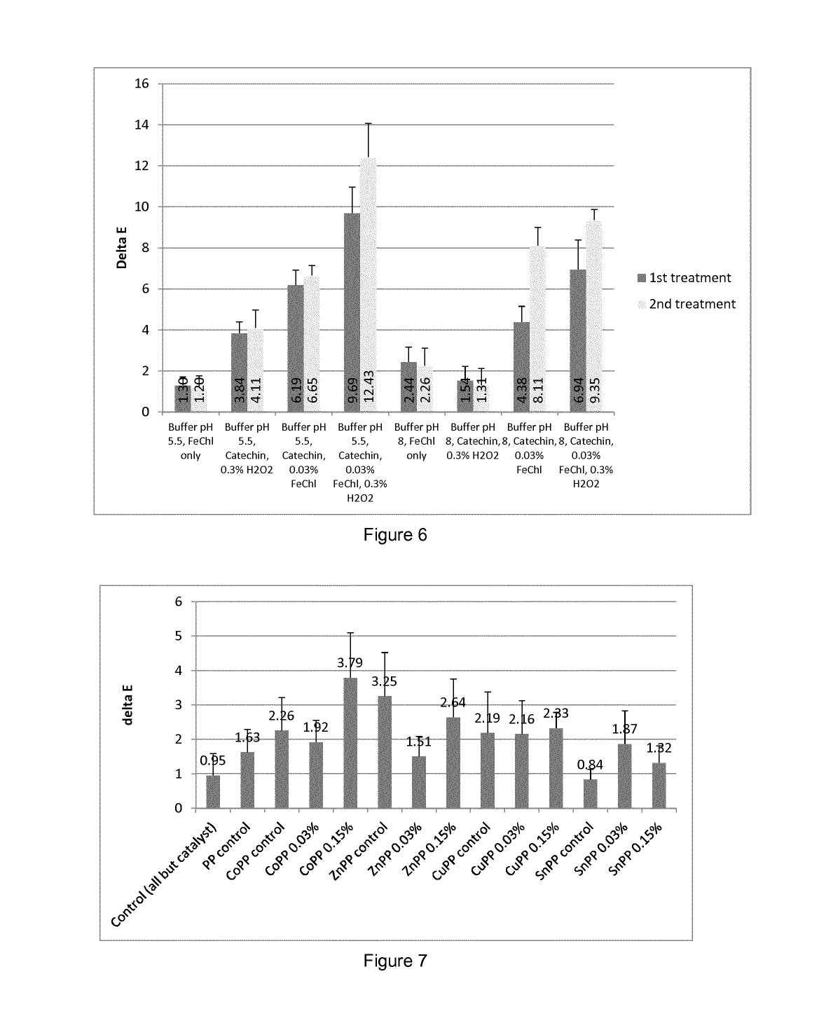

The results are presented in FIG. 6 and show that change in colour is lower at pH 8.0 than at pH 5.5.

### Example 6

### Materials

Cobalt (II) protoporphyrin (Sigma Aldrich)

Zinc (II) protoporphyrin (Sigma Aldrich)

Copper (II) protoporphyrin (Santa Cruz Biochemicals)

Tin (II) protoporphyrin (Santa Cruz Biochemicals)

Protoporphyrin IX (Sigma Aldrich)

### Method

The method was identical to that set forth in Example 1 except only one rather than five treatments with the hair colour composition were performed. The hair colour compositions with cobalt (II) protoporphyrin are set forth in table 5.

TABLE 5
Hair colour compositions. Total volume in all reactions was
6000 μl and therefore the initial concentrations for each of the
ingredients (where present) was 17.6 mM catechin, 0.03 or 0.15% w/w
cobalt (II) or 0.3% protoporphyrin IX, and 0.3% w/w hydrogen peroxide.

| | Volume | Volume | Volume | Volume | Volume |
|---|---|---|---|---|---|
| Ingredient | (μl) | (μl) | (μl) | (μl) | (μl) |
| Catechin (50 mg/ml) | 600 | 600 | 600 | 600 | 600 |
| Cobalt (II) | 0 | 0 | 600 | 600 | 0 |
| protoporphyrin | | | | | |
| (0.3% w/w) | | | | | |
| Cobalt (II) | 0 | 0 | 0 | 0 | 600 |
| protoporphyrin | | | | | |
| (1.5% w/w) | | | | | |
| Protoporphyrin IX | 600 | | | | |
| H₂O₂ (3% w/w) | 600 | 600 | 600 | 0 | 600 |
| 50 mM Britton Robinson | 4200 | 4800 | 4200 | 4800 | 4200 |
| buffer pH 5.5 | | | | | |

The hair colour compositions with the remaining metal protoporphyrins followed the same pattern as set forth in table 5.

### Results

The results are presented in FIG. 7 and show that no significant change in colour was achieved except possibly with cobalt (II) protoporphyrin.

### Example 7

### Method

The test with cobalt (II) protoporphyrin was repeated at a final concentration of 0.03% w/w in the manner described in Example 6 but with two treatments.

### Results

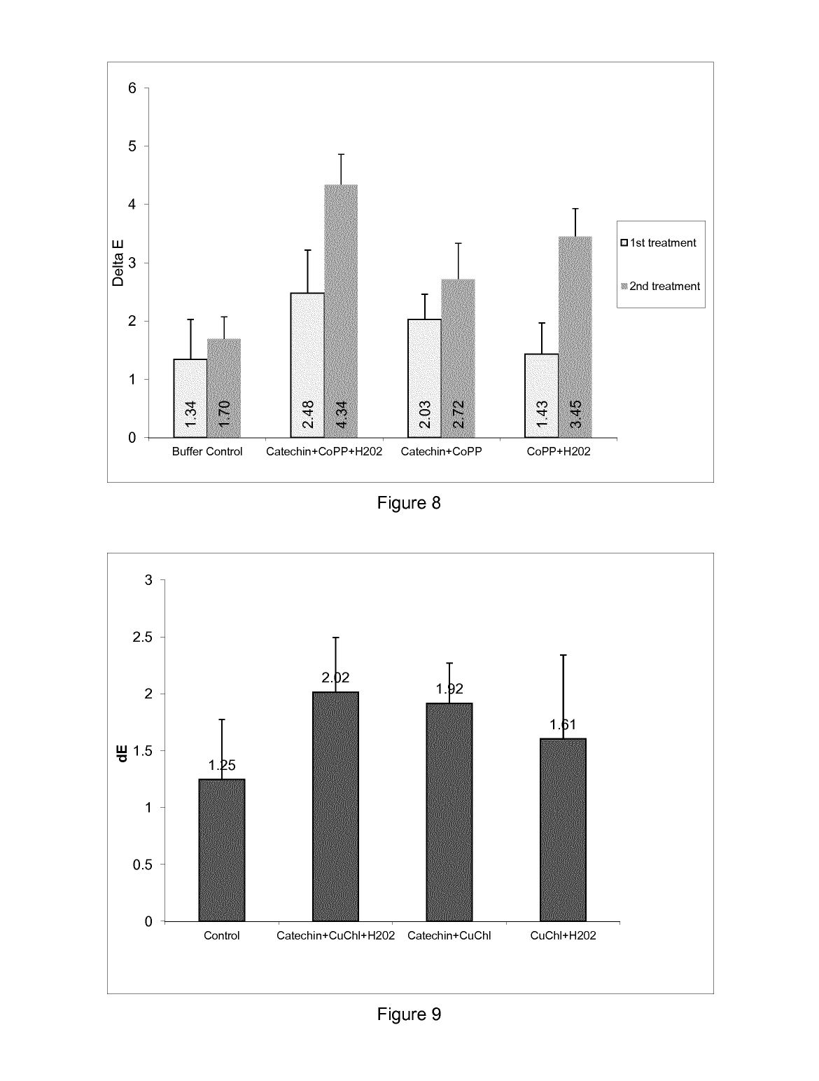

The results are presented in FIG. 8 which show that treatment of hair switches with the hair colour composition based on cobalt (II) protoporphyrin does lead to a change in hair colour.

### Example 8

### Materials

Copper chlorophyllin (Sigma Aldrich)

### Method

The method was identical to that set forth in Example 3 except only a single treatment with the hair colour composition was performed.

### Results

The results are presented in FIG. 9 and show that no significant change in colour was achieved.

### Example 9

### Materials

Magnesium chlorophyll A (Sigma Aldrich)

### Method

The method was identical to that set forth in Example 3 except only a single treatment with the hair colour composition was performed.

### Results

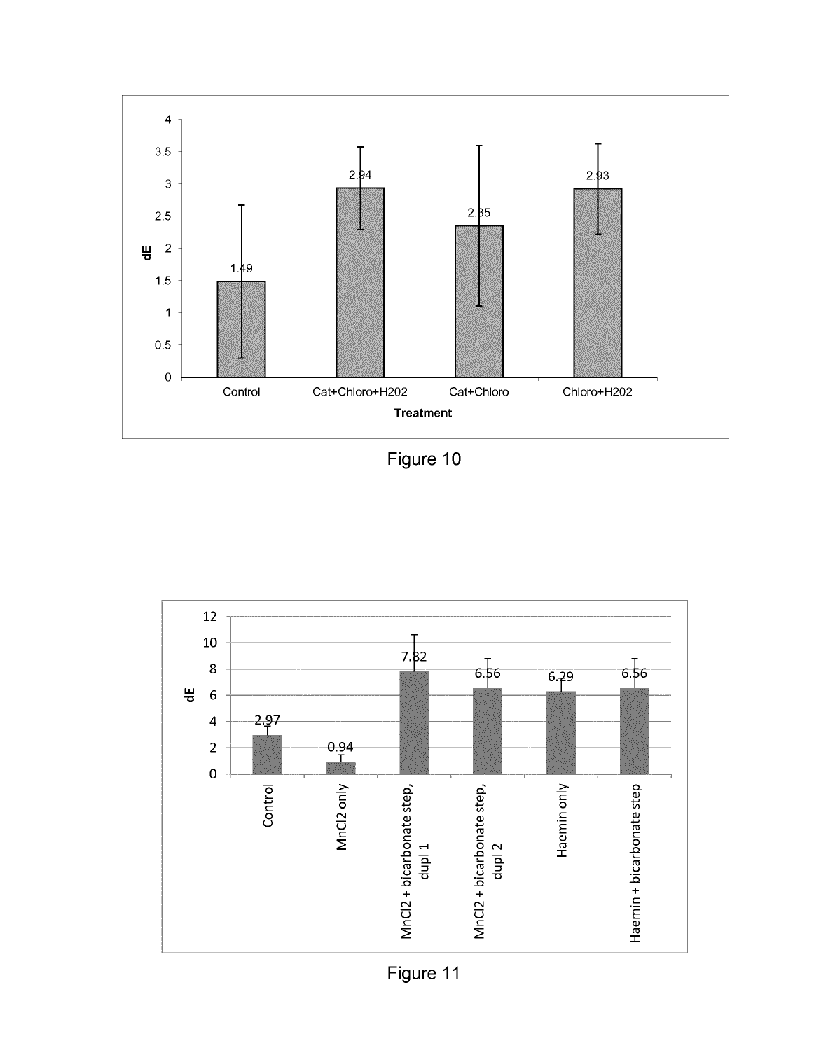

The results are presented in FIG. 10 and show that no significant change in colour was achieved.

### Example 10

### Materials

Manganese chloride tetrahydrate (Sigma Aldrich)

Sodium bicarbonate (Sigma Aldrich)

Carbopol 934 (Lubrizol Advanced Materials, USA)

### Method

The method was identical to that set forth in example 1 except that only one rather than five treatments with the hair colour composition were performed, and in three of the treatments with composition A, prior to the shampooing step there was an intermediate step of a 10 minute incubation of the drained hair switch in composition B containing sodium bicarbonate. The hair colour compositions are set forth in table 6.

TABLE 6
Hair colour compositions. Total volume in all composition A reactions was
6000 μl and therefore the initial concentrations for each of the ingredients (where
present) was 0.5% catechin, 1.82 mM manganese chloride tetrahydrate or 0.46 mM
haemin, and 0.35% w/w hydrogen peroxide. Total volume in composition B reactions
was 4000 μl.

| Ingredient (%) | 1 | 2 | 3 | 4 | 5 | 6 |
|---|---|---|---|---|---|---|
| Composition A | | | | | | |
| Catechin | 0 | 0.5 | 0.5 | 0.5 | 0.5 | 0.5 |
| Manganese chloride | | 0.036 | 0.036 | 0.036 | 0 | 0 |
| tetrahydrate | | | | | | |
| Haemin | 0 | 0 | 0 | 0 | 0.03 | 0.03 |
| H₂O₂ | 0.3 | 0.3 | 0.3 | 0.3 | 0.3 | 0.3 |
| Britton Robinson buffer | To 100 | To 100 | To 100 | To 100 | To 100 | To 100 |
| pH 5.5 | | | | | | |
| | | | Second | Second | | Second |
| | | | step | step | | step |
| Composition B | | | | | | |
| Sodium bicarbonate | | | 2.6 | 2.6 | | 2.6 |
| Carbopol 934 | | | 1 | 1 | | 1 |
| Ethanolamine | | | To pH 9.0 | To pH 9.0 | | To pH 9.0 |
| Deionised water | | | To 100 | To 100 | | To 100 |

### Results

The results are presented in FIG. 11 and show that no significant change in colour was achieved with magnesium chloride tetrahydrate without treatment with a sodium bicarbonate solution at pH 9.0. The performance of the two part hair colour composition comprising magnesium chloride tetrahydrate without treatment with a sodium bicarbonate solution at pH 9.0 was not significantly better than the single part hair colour composition based on haemin at pH 5.5 even though there was four times more, in molar terms, magnesium chloride tetrahydrate.

1. A hair colour composition comprising a flavonoid, a catalyst comprising iron (II) or cobalt (II) complexed to a non-proteinaceous heterocyclic macrocycle ligand composed of pyrrole or pyrroline subunits, and either a hydrogen peroxide generator or 0.0001 to less than 1.5% w/w hydrogen peroxide;
wherein the flavonoid is selected from the group consisting of flavones, isoflavones, flavans, isoflavans, flavanones, flavonols, flavan-3-ols, dihydroflavonol, flavanonols, proanthocyanidins, aurones, chalcones, dihydrochalcones, flavonolignans, and derivatives thereof,
wherein the hair colour composition has a pH of 4.5 to 8.0.

2. A hair composition according to claim 1,
wherein the flavone is selected from the group consisting of apigenin, luteolin and chrysin;
wherein the isoflavone is selected from the group consisting of daidzein, genistein, formononetin and orobol;
wherein the flavan is 4′-hydroxy-5,6-dimethoxyflavan;
wherein the isoflavan is glabridin or licoricidin;
wherein the flavanone is eriodictyol or naringenin;
wherein the flavonol is selected from the group consisting of myricetin, kaempferol, quercetin and gossypetin;
wherein the flavan-3-ol is selected from the group consisting of catechin, theaflavin, gallocatechin, catechin gallate, gallocatechin gallate, epicatechin, epigallocatechin, epigallocatechin gallate and epigallocatechin gallate;
wherein the dihydroflavonol is taxifolin or aromadendrin;
wherein the aurone is sulphuretin;
wherein the chalcone is 2′-hydroxy-4-methoxy-chalcone;
wherein the dihydrochalcone is phloretin or phloridzin and
wherein the flavonolignan is silibinin or silichristin.

3. A hair composition according to claim 1 wherein the non-proteinaceous heterocyclic macrocycle ligand composed of pyrrole or pyrroline subunits is selected from the group consisting of protoporphyrins, corrins, porphyrins, porphorinogens, chlorins bacteriochlorins, isobacteriochlorins, corphins and corrole.

4. A hair composition according to claim 1 wherein the catalyst is selected from the group consisting of cobalamins, haemin, Haem A, Haem B, Haem C, Haem 0, iron (II) chlorophyllin and siroheme.

5. A hair composition according to claim 1 wherein the catalyst is selected from the group consisting of haemin, sodium iron chlorophyllin and cobalt (II) protoporphyrin.

6. A hair composition according to claim 1 comprising 0.001-10, preferably 0.01-5, most preferably 0.1-2% w/w flavonoid.

7. A hair composition according to claim 1 comprising 0.001-1, preferably 0.01-0.5% w/w hydrogen peroxide.

8. A hair composition according to claim 1 comprising 0.0001-5, preferably 0.001-1% w/w catalyst.

9. A hair composition according to claim 1, wherein the hydrogen peroxide generator comprises a hydrogen peroxide generating oxidase, a substrate and oxygen.

10. A hair composition according to claim 1 wherein the hair composition is a shampoo and/or a conditioner.

11. A kit for colouring hair comprising:
(a) A hair composition according to claim 1; and
(b) A pre- or post-treatment composition comprising a metal ion suitable for coordinating to the flavonoid or the product of the reaction of the flavonoid in the presence of the catalyst.

12. A kit according to claim 11, wherein the metal ion is selected from the group consisting of iron (II), iron (III), copper (I), copper (II), copper (III), aluminium (III), zinc (I), zinc (II), manganese (II), manganese (III), manganese (IV), manganese (V), manganese (VI) and manganese (VII).

13. A kit according to claim 11 wherein the pre- or post-treatment composition is a shampoo and/or a conditioner.

14. A method for colouring hair comprising the step of treating hair with the hair colour composition of claim 1.

15. A method according to claim 14 comprising the additional steps of:
(a) treating the hair with the pre- or post-treatment composition of claim 11; and
(b) washing the hair;
wherein step (a) is completed prior to or after treatment of the hair with the hair composition; and
wherein step (b) is completed between step (a) and treatment of the hair with the hair composition.
